(19) United States
(12) Patent Application Publication
(10) Pub. No.: US 2016/0178589 A1
(43) Pub. Date: Jun. 23, 2016

(54) SYSTEM AND METHOD OF DISPLAYING GAS CONCENTRATIONS

(71) Applicant: Honeywell International Inc., Morristown, NJ (US)

(72) Inventors: Shashikant G. Gulaguli, Bangalore (IN); Gowrisankar M R, Bangalore (IN); Anupama K. Ravi, Bangalore (IN); Irfan K, Bangalore (IN); Anand Tyagaraj, Bangalore (IN); Vijayapavan Amaravadi, Hyderabad (IN); Jose R. Diaz, Glendale Heights, IL (US)

(21) Appl. No.: 14/581,318

(22) Filed: Dec. 23, 2014

(51) Int. Cl. G01N 33/00

(52) U.S. Cl. CPC G01N 33/0063

(57) ABSTRACT

An apparatus and method to present developing incidents to a user in accordance with a selected criteria. Where the incidents correspond to developing gas concentrations, gas samples can be compared to a selected threshold, and responsive to the comparison, a concentration indicating display, relative to a selected region being monitored, can be presented to a user. The displayed concentration indicating elements, in response to real-time samples, can expand on the visual presentation indicating the characteristics of a developing incident. Stored historical information can be represented for after action analysis.

### FIELD

The application pertains to monitoring systems and methods of managing large numbers of gas detectors used in monitoring regions of interest. More particularly, the application pertains such systems and methods which provide graphical displays of gas concentrations nearing low alarm set points. Real time or historical concentrations and trends can be displayed along with incident location information.

### BACKGROUND

Industrial processing facilities, such as oil & gas refineries, natural gas fields and the like, will normally be equipped with fixed gas detectors located across facility. Workers also will be carrying personal gas detectors & roaming across the facility as part of their normal work schedule

When the gas concentrations go beyond an alarm set point, then both fixed & personal gas detectors provide notification and such incidents will be attended to. However, when stray gas incidents are building up those concentrations, they may not build up to an alarm set point for a while. Instead, such data samples will be just logged by various of the gas detectors and may go unnoticed.

Such stray gas incidents, which might be ignored, may result in major gas leakages if not promptly attended to.

Unfortunately, at times, there is no automation/notification mechanism available to inform about presence of stray gas leakage. Further, neither fleet management, nor a safety manager may look at data logs collected by gas detectors on a regular basis.

One current process is to view gas readings from gas detectors in a fleet management application. It is difficult/error prone to correlate incident locations with data samples collected by the gas detectors.

Known real time monitoring applications do not necessarily provide any indication about such low concentration level events. Concentrations may be displayed only on demand. Such systems may continue to display device status as normal.

Alarm set points vary for different gas types and it is challenging to distinguish boundaries between normal and low alarm values

### BRIEF DESCRIPTION OF THE DRAWINGS

FIG. 1 illustrates a block diagram of a system in accordance herewith;

FIG. 2 illustrates a diagram of a process in accordance herewith; and

FIGS. 3-10 illustrate user interface displays presenting a sequence of detected relatively low gas concentrations so that the respective incident can be addressed and corrective action taken.

### DETAILED DESCRIPTION

While disclosed embodiments can take many different forms, specific embodiments thereof are shown in the drawings and will be described herein in detail with the understanding that the present disclosure is to be considered as an exemplification of the principles thereof as well as the best mode of practicing same, and is not intended to limit the application or claims to the specific embodiment illustrated.

In one aspect, data samples can be collected from portable and/or fixed gas detectors and analyzed for the presence of gas concentrations nearing respective low alarm set points. Safety personnel as well as management can be automatically notified as to the presence of such events and the corresponding locations where such samples are being collected.

Incidents, in yet another aspect, can be displayed on one or more local displays, at the facility, or, central monitoring station. Advantageously, sequential displays of developing, relatively low, concentrations can be visually presented locally or remotely, in real-time or in selected time intervals. Historical concentration information, along with location information can also be stored and presented visually.

In yet another aspect, data points indicative of increasing concentrations can be displayed as multi-part elements. One element can be proportional to reported concentration, for example with circularly presented data points, an outer circular portion having a first diameter, can be proportional to sensed or reported concentration at a selected detector. An inner circular region with a smaller diameter can indicate concentration proportional to a respective low alarm set point.

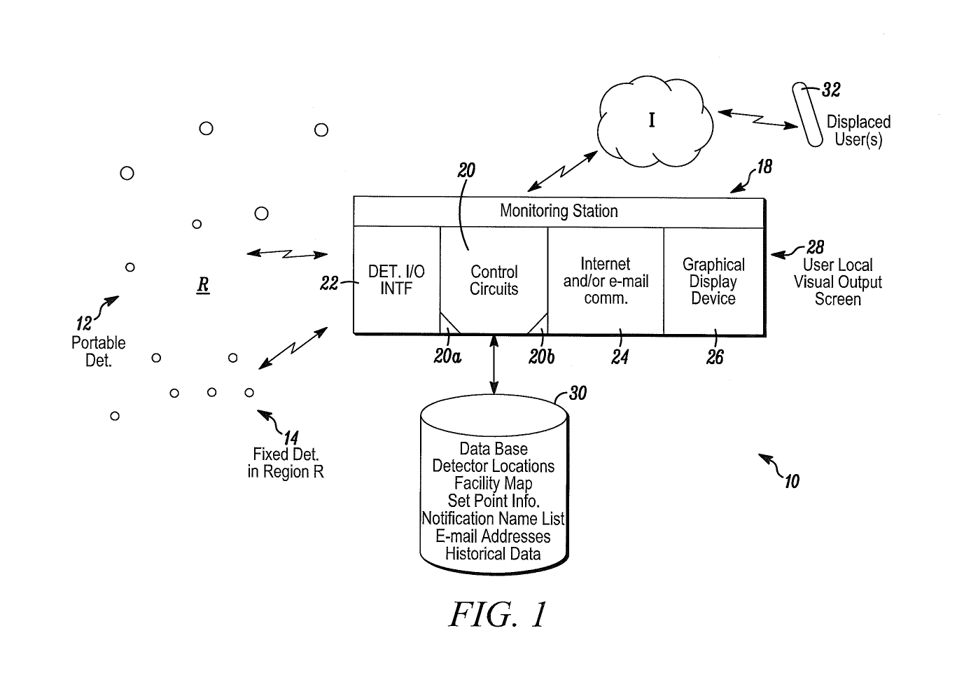

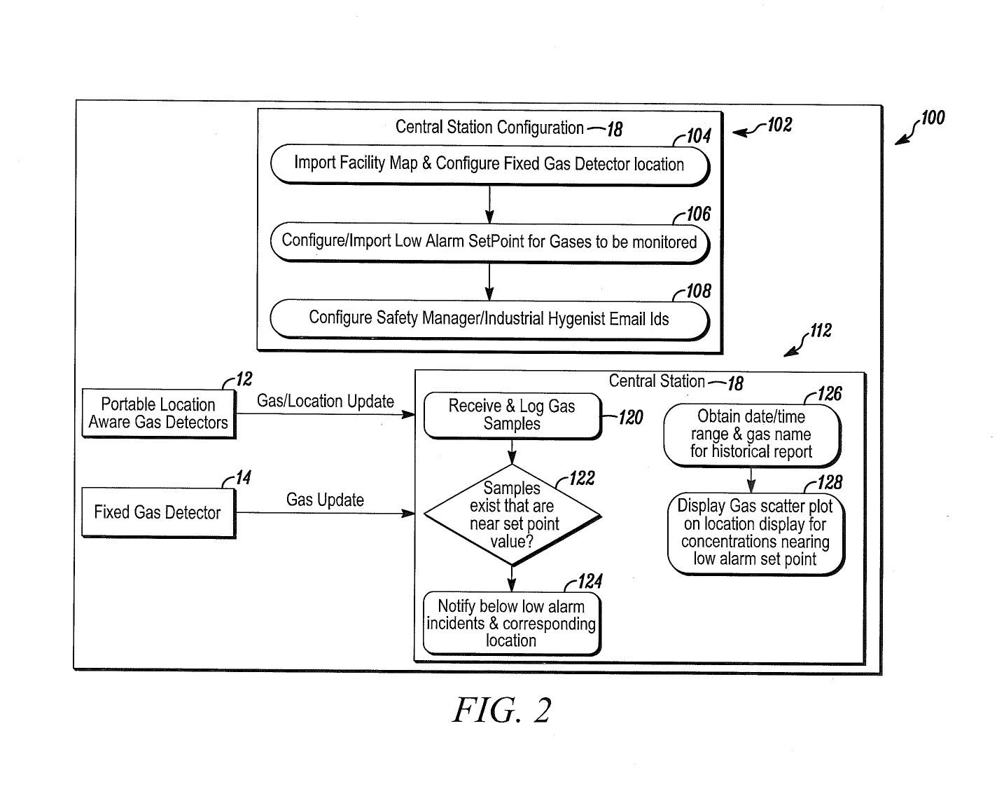

FIGS. 1, 2 taken together illustrate aspects of a system 10 and method 100 in accordance herewith. Exemplary system 10 includes a plurality of portable gas detectors 12 in a region R being monitored. Fixed gas detectors 14 can also be distributed throughout the region R. Neither the types of gas nor the types of detectors in region R of system 10 are limitations hereof.

Members of the pluralities 12, 14 can be in wired or wireless communication with a monitoring station 18. Station 18 can provide monitoring services from a site local to the region, or facility R. Alternately, where the detectors 12, 14 are internet enabled, station 18 can be displaced from the region R.

Exemplary station 18 can include control circuits 20 which are coupled to a gas detector interface 22. The control circuits 20 can be implemented at least in part by one or more programmable processors 20a which execute stored control, or, analysis software 20b.

Station 18 can also include Internet and/or E-mail communications interfaces 24, coupled to control circuits 20. One or more user oriented input/output devices, such as graphical display devices 26 can receive user input from and provide graphical, visual user outputs via interface 28. Data can be collected in storage unit 30 in a data base for use in further processing, displaying developing incidents or generating historical reports.

Station 18 can also be wirelessly enabled and provide visual communications via a communications device 32, to displaced users via computer networks, such as an intranet, or, internet.

Aspects of process 100 are illustrated in FIG. 2 which includes reference to elements of system 10. Station 18 can be configured, as at 102, by importing facility map(s) and configuration information relative to fixed gas detector locations, as at 104.

Low alarm gas set point information can be imported as at 106. Additionally, safety management information can be imported as at 108. Such information can identify local, or displaced safety or management personnel who should receive notices, or graphical outputs of developing incidents.

Operational aspects, as at 112 include receiving and logging incoming gas samples at station 18, as at 120. Samples can be analyzed to determine if some or all of them are near a predetermined set point value, as at 122. Low concentrations that fall below low alarm thresholds or set points can be identified along with corresponding location, as at 124.

Historical reports can be created based on incoming samples, as at 126. Further, possible incident indicating scatter plots associated with concentrations that are below, but might be nearing a low alarm set point can be created and displayed, as at 128. Time based sequences of developing incidents can thus be made available to safety personnel.

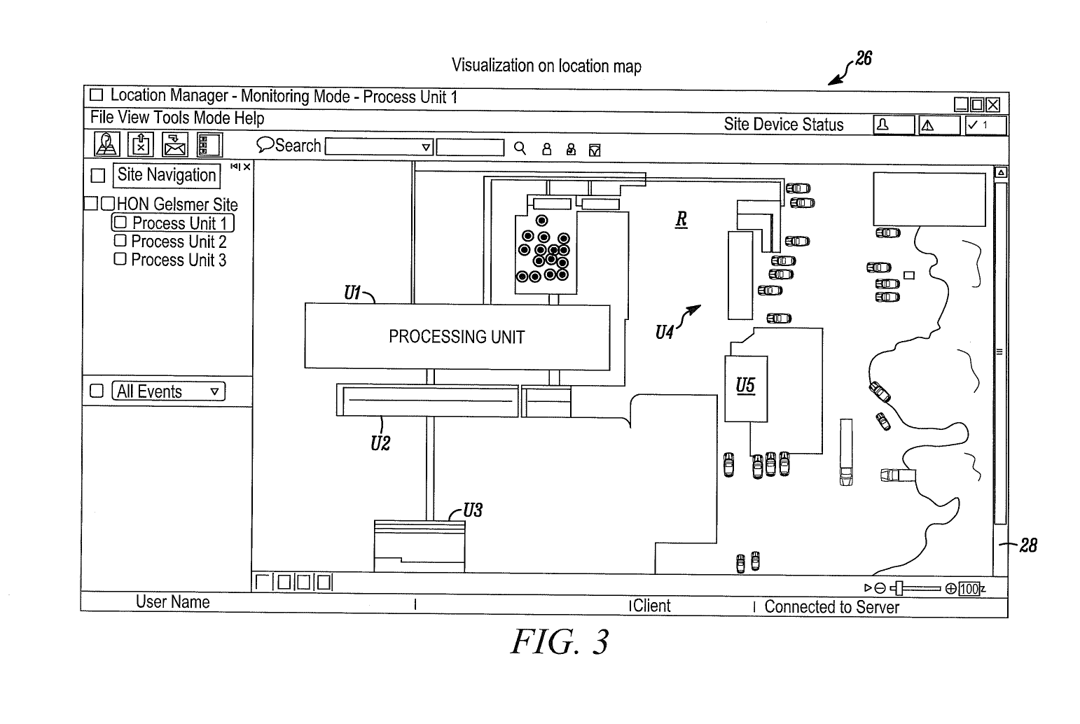

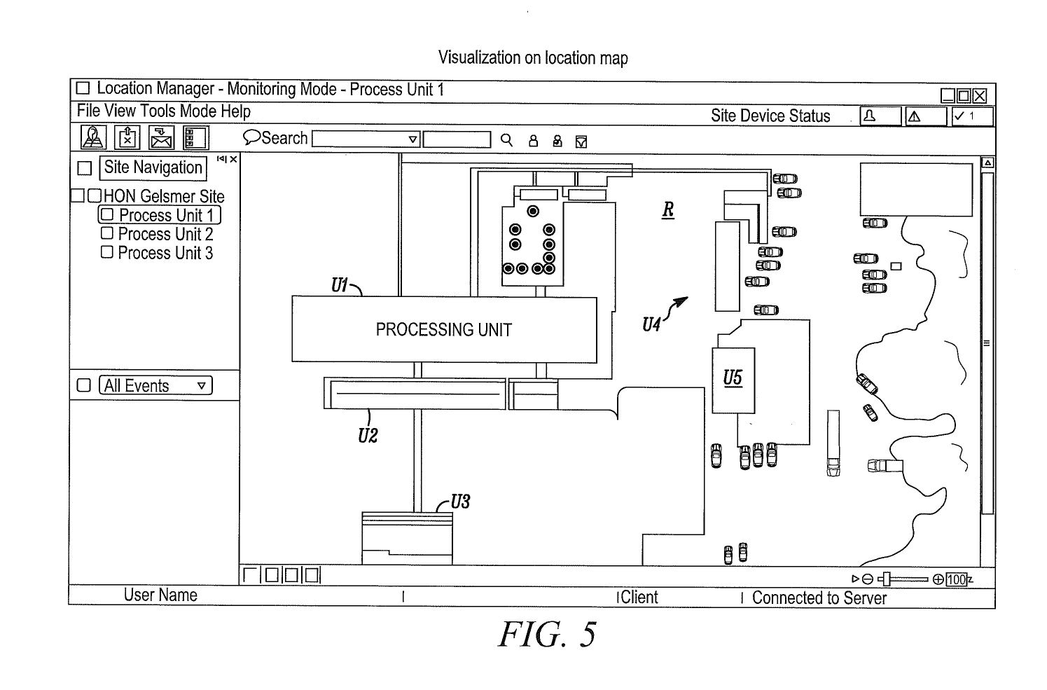

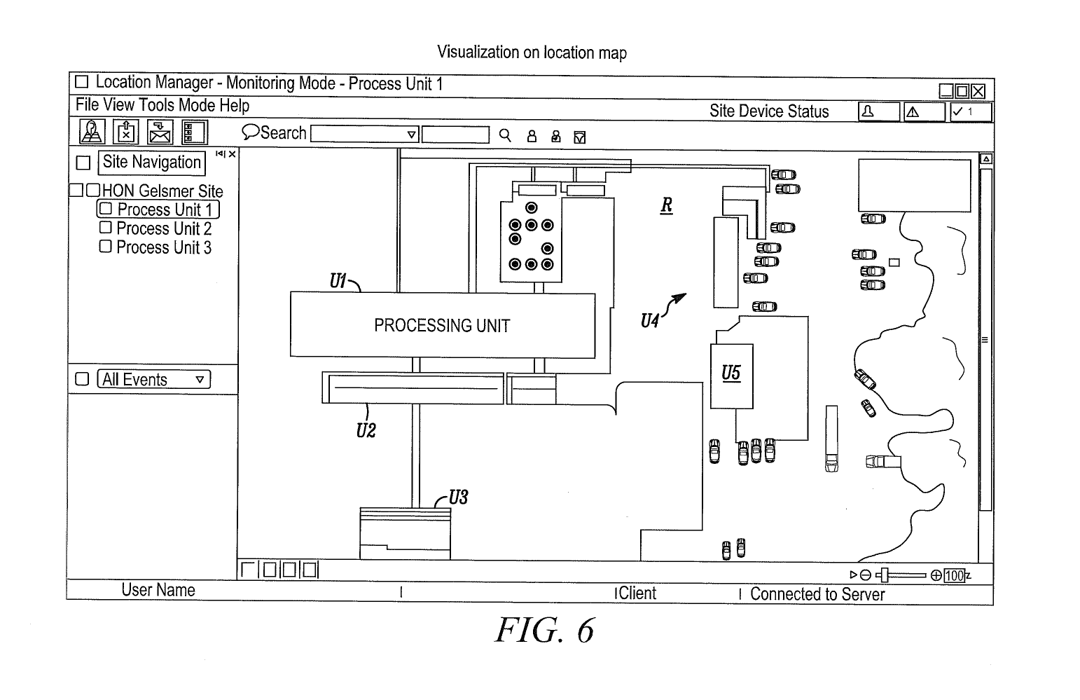

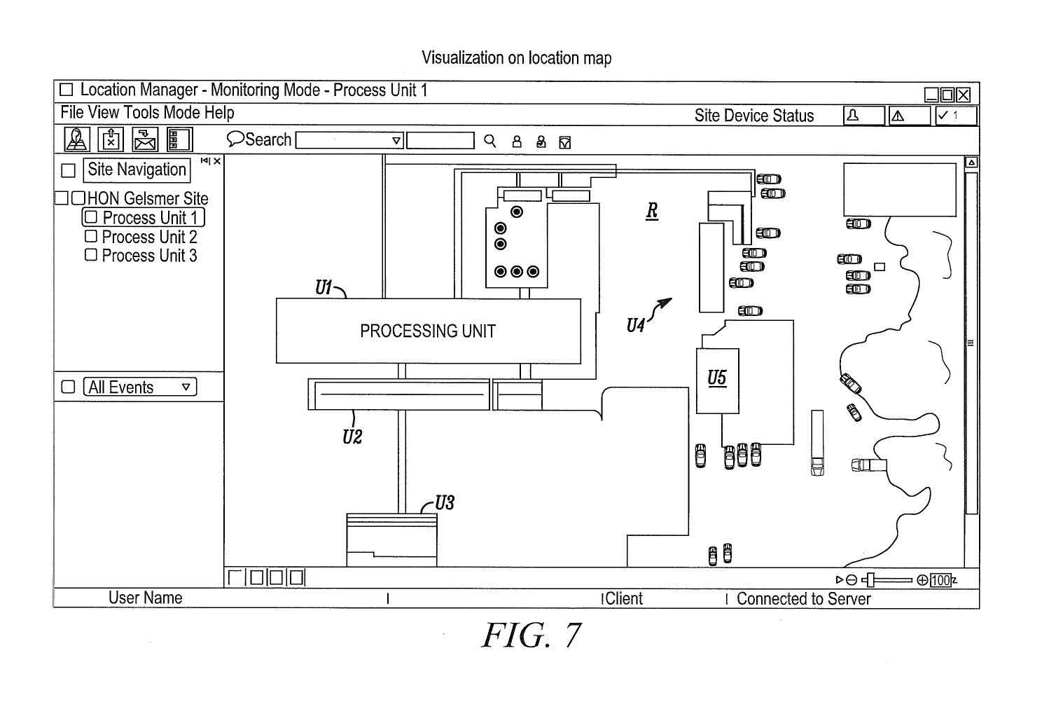

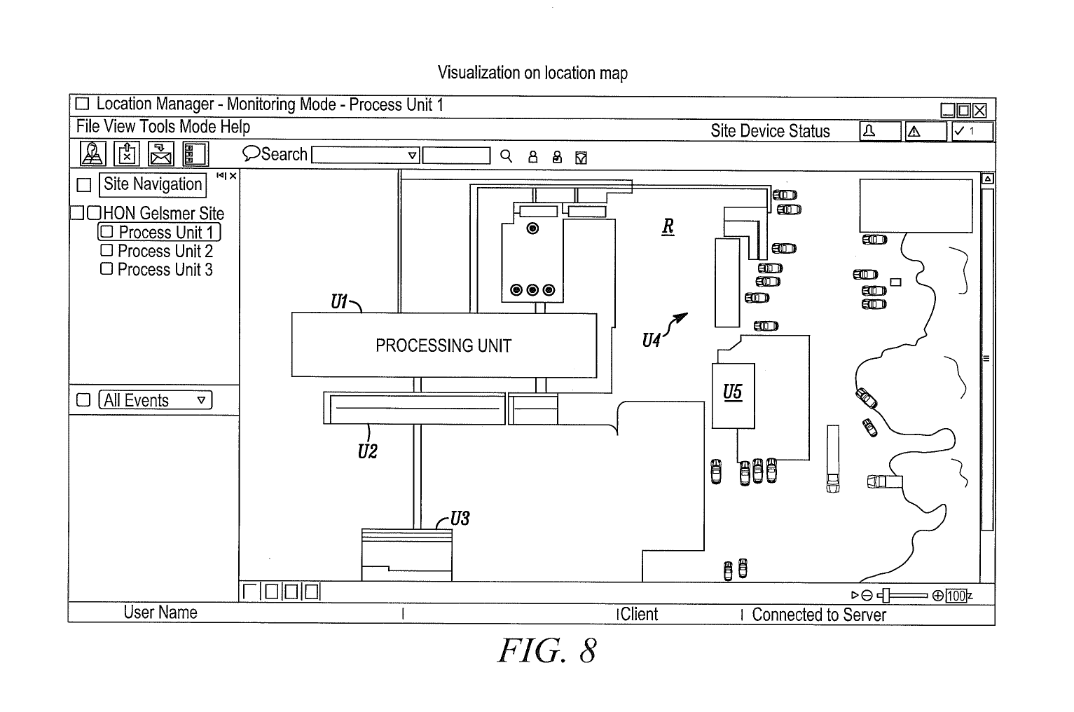

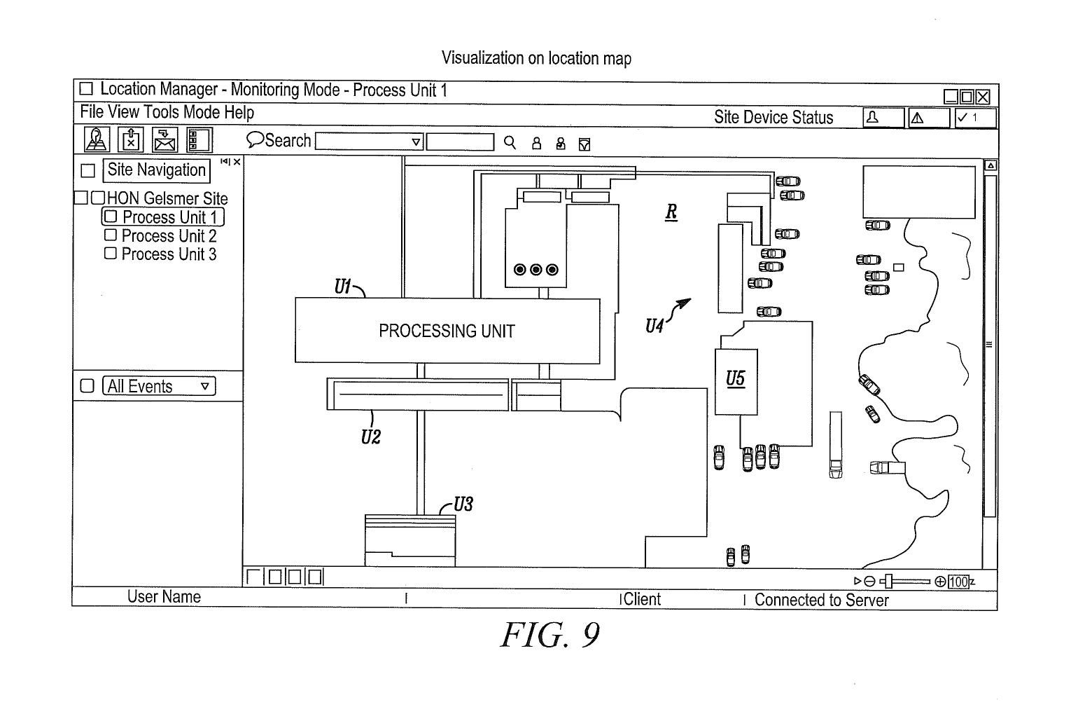

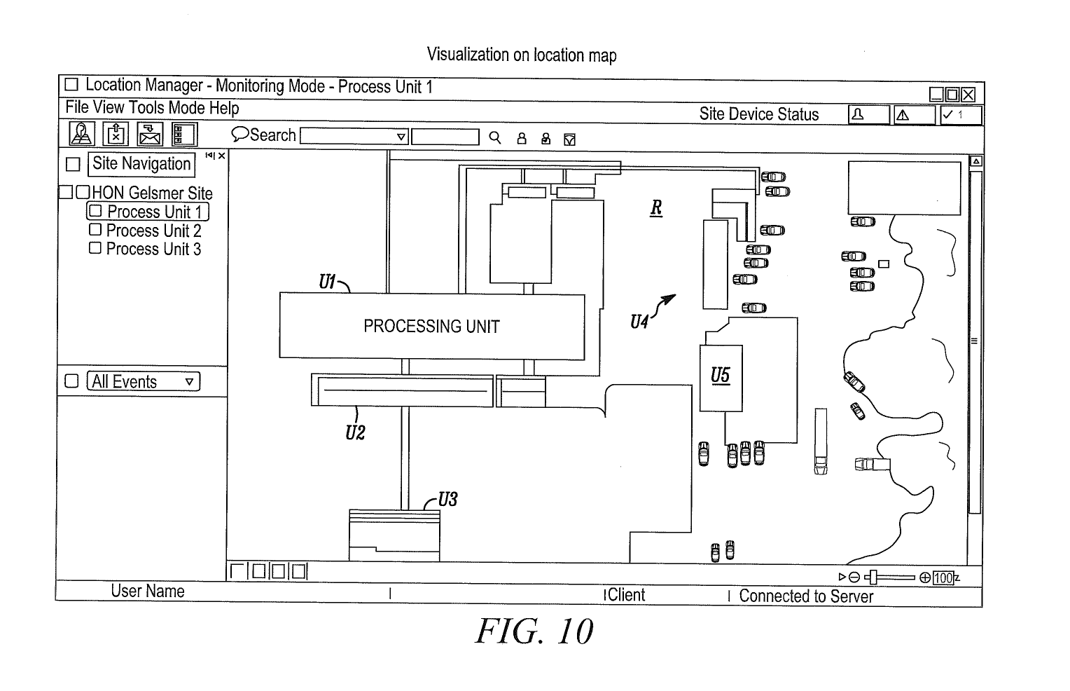

FIGS. 3-10 illustrate a facility, with one or more processing units that is presented on a visual output 28 of a display unit 26. Detected gas concentration locations, as at 128, are presented on the displays of FIGS. 3-10 as an incident develops. It will be understood that the type of facility is not a limitation hereof.

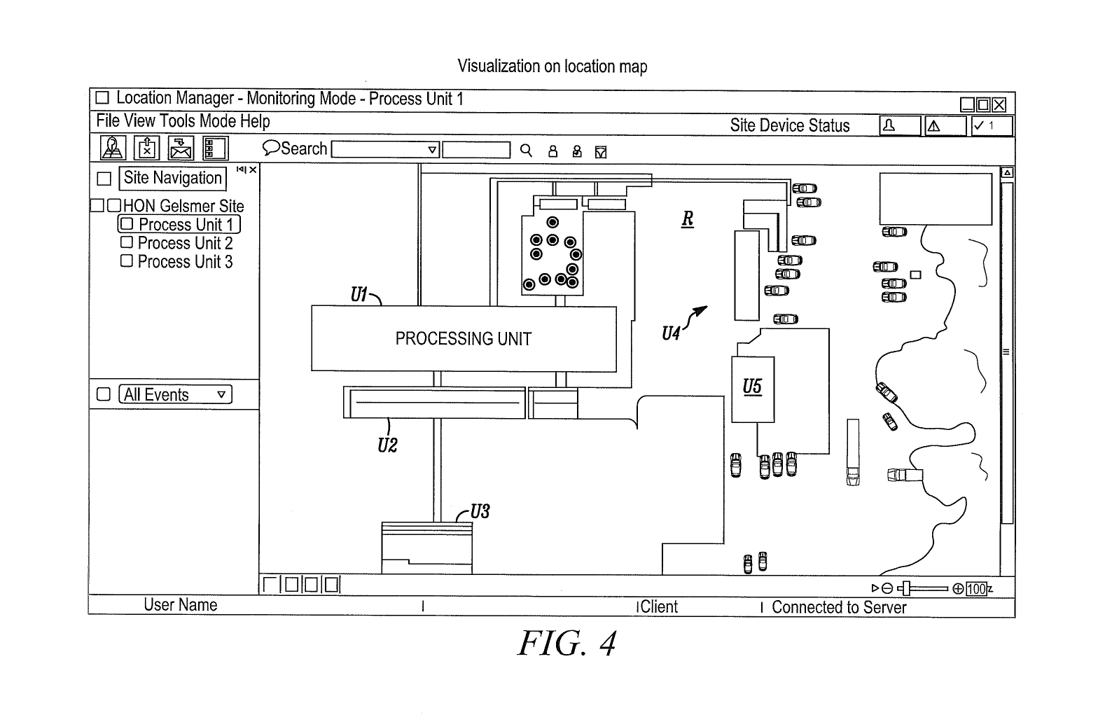

In each of FIGS. 4-10 developing low level concentrations of a selected gas are presented as a function of time as the incident is developing. As can be seen, safety or management personnel can dynamically view these low level developing incidents, show by an increasing number of detectors with concentrations approaching a pre-established set point.

Each of the activated detectors can be presented as a circular element. A single circle can have a diameter indicative of a current sensed gas concentration. The center of the circle identifies the detector's location. Multi-element indictors are illustrated in FIGS. 4-10. An outer circle has a diameter proportional to a sensed, or reported concentration. An inner circle has a diameter promotional to a low alarm set point.

In summary, circular images of different diameters can be presented indicating different gas concentrations. The center of each such image is located on the display where the gas concentration sample has been obtained. It will also be understood that concentration indicating images are not limited to circular elements. For example, triangular or square elements could also be used without departing from the spirit and scope hereof.

From the foregoing, it will be observed that numerous variations and modifications may be effected without departing from the spirit and scope hereof. It is to be understood that no limitation with respect to the specific apparatus illustrated herein is intended or should be inferred. It is, of course, intended to cover by the appended claims all such modifications as fall within the scope of the claims. Further, logic flows depicted in the figures do not require the particular order shown, or sequential order, to achieve desirable results. Other steps may be provided, or steps may be eliminated, from the described flows, and other components may be add to, or removed from the described embodiments.

1. A method comprising:
providing a plurality of gas detectors;
receiving gas concentration information from at least some of the detectors, along with location information;
evaluating received concentration information, and determining if samples have been received which are near a selected set point;
providing visual display elements where the elements represent different detected gas concentrations which are presented with at least one of color related or shape related features.

2. A method as in claim 1 which includes providing at least one detector display element which includes first and second parts indicative of first and second detector conditions.

3. A method as in claim 1 which includes dynamically updating the presented display elements at a predetermine sample rate.

4. A method as in claim 3 where altering the display elements provides an indication of changing gas conditions.

5. A method as in claim 4 which includes presenting at least one detector display element which includes first and second parts indicative of first and second detector conditions.

6. A method as in claim 1 which includes presenting a plurality of detector display elements some of which include first and second parts indicative of first and second detector conditions.

7. A method as in claim 4 which includes storing a sequence of dynamically changing, displayed elements.

8. A method as in claim 7 which includes retrieving and representing stored display sequences.

9. An apparatus comprising:
a display device;
a storage element;
control circuits coupled to the display device and the storage element; and
interface circuits coupled to the control circuits, wherein, gas concentration and location information obtained from members of a plurality of gas detectors is coupled, via the interface, to the control circuits to be presented on the display device as a plurality of location and concentration indicating symbols.

10. An apparatus as in claim 9 where received concentrations are compared to a pre-determined threshold value stored in the storage element.

11. An apparatus as in claim 10 wherein indicating symbols are only displayed where the respective concentrations approach the predetermined threshold.

12. An apparatus as in claim 10 where the control circuits sample received concentrations at a predetermined rate, and store each sample sequence in the storage element.

13. An apparatus as in claim 12 which includes a manually operable control element to select a view of currently presented concentration symbols, or a sequence of pre-stored samples.

14. An apparatus as in claim 9 where concentration indicating symbols comprise one of circles, squares, or, triangles.

15. An apparatus as in claim 9 where concentration indicating symbols comprise a dual characteristic indicating element.

16. An apparatus as in claim 15 with the symbols comprising first and second concentric shapes.

17. An apparatus as in claim 16 where an inner symbols represents a first data type and an outer symbol represent a second data type.

18. An apparatus as in claim 17 where each symbol discloses location, concentration and set-point information.

19. An apparatus as in claim 15 where indicating elements comprise one of multi-coded, pie shaped, circular or tear shaped sub-elements.
